(19) United States
(12) Patent Application Publication
(10) Pub. No.: US 2026/0240806 A1
(43) Pub. Date: Aug. 20, 2026

(54) R-MDMA and S-MDMA TO ASSIST PSYCHOTHERAPY

(71) Applicant: Universitätsspital Basel, Basel (CH)

(72) Inventor: Matthias Emanuel LIECHTI, Basel (CH)

(21) Appl. No.: 19/642,435

(22) Filed: Apr. 8, 2026

Related U.S. Application Data
(63) Continuation of application No. 18/065,665, filed on Dec. 14, 2022.
(60) Provisional application No. 63/297,322, filed on Jan. 7, 2022.

(51) Int. Cl. A61K 31/357; A61P 25/30

(52) U.S. Cl. CPC A61K 31/357; A61P 25/30

(57) ABSTRACT

A composition including R-MDMA, S-MDMA, or specific (not 1:1 as in racemic MDMA) combinations of these two enantiomers of racemic MDMA as well as R-MDA, S-MDA, and a combination that is not 1:1 of R-MDA and S-MDA. A method of treating an individual, especially in substance-assisted psychotherapy by administering the composition to the individual. A method of personalized medicine, by evaluating an individual who is in need of MDMA treatment and determining if there are characteristics of the individual present that would not be suitable for MDMA treatment, and administering the composition to the individual. A method of reducing abuse of MDMA by an individual, by administering R-MDMA to the individual and thereby reducing abuse.

### CROSS-REFERENCE TO RELATED APPLICATIONS

This application is a continuation of U.S. application Ser. No. 18/065,665, filed Dec. 14, 2022, which claims priority to, and the benefit of, U.S. Provisional Application No. 63/297,322, filed Jan. 7, 2022, the contents of which are incorporated by reference in their entirety.

### BACKGROUND OF THE INVENTION

### 1. Field of the Invention

The present invention relates to applications of the R- and S-enantiomers of 3,4-methylenedioxymethamphetamine as treatments for medical conditions.

### 2. Background Art

Racemic ±3,4-methylenedioxymethamphetamine (MDMA) is a psychoactive substance and prototypical empathogen acutely inducing feelings of heightened mood, empathy, trust and closeness to others (Hysek et al., 2014a). These acute subjective effects of MDMA may be helpful to assist psychotherapy and MDMA is currently investigated in phase 3 trials as a possible treatment of post-traumatic stress disorder (PTSD) (Mitchell et al., 2021).

MDMA is a racemic substance containing equal amounts of the enantiomers S(+)- and R(−)-MDMA. At present the enantiomers of MDMA have not been investigated in humans.

Preclinical research indicates that S-MDMA mainly releases dopamine (DA), norepinephrine (NE), serotonin (5-HT), and oxytocin while R-MDMA may act more directly on serotonin 5-HT2A receptors and release prolactin (PRL). Animal studies also indicate that the two enantiomers act synergistically to produce the subjective effects of MDMA and that S-MDMA is mainly responsible for psychostimulation while R-MDMA may have fewer adverse effects and have greater prosocial effects. However, acute effects of S- and R-MDMA have never been validly examined in a human study.

MDMA is currently being investigated in patients with PTSD, social anxiety, autism (Danforth et al., 2018; Danforth et al., 2016; Mithoefer et al., 2019; Mithoefer et al., 2010; Oehen et al., 2013), and may later also be studied and used for a range of other medical conditions. Such conditions where MDMA or R-MDMA or S-MDMA or combinations of R-and S-MDMA can be useful include, but is not limited to, substance-use disorder, depression, anxiety disorder, anxiety with life-threatening disease, personality disorder including narcistic and antisocial disorder, and obsessive-compulsive disorder. R-or S-MDMA or combinations thereof can also be used to enhance couple therapy.

MDMA and related substances are thought to produce positive therapeutic long-term effects in the context of MDMA/substance-assisted psychotherapy by producing acute subjective positive mood effects that also enhance the effectiveness of psychotherapy and can be beneficial on their own. Such acute beneficial MDMA-effects include, but are not limited to, feelings of well-being, feelings of connectivity to others, feelings of increased trust, feelings of love, enhanced emotional empathy, and enhanced feelings of pro-sociality and prosocial behavior (Dolder et al., 2018; Holze et al., 2020; Hysek et al., 2014a; Schmid et al., 2014).

Prior art discloses the use of MDMA in substance-assisted psychotherapy (Mitchell et al., 2021; Mithoefer et al., 2010; Oehen et al., 2013). However, R-and S-MDMA or specific combinations thereof can be more suitable with different therapeutic benefits/tolerability profiles. R-and S-MDMA have different actions than racemic MDMA as is further explored in the study describe in the EXAMPLE.

Amphetamines including MDMA carry a certain risk of abuse liability. This is evidenced by the fact that MDMA is self-administered by animals (Cole & Sumnall, 2003; Creehan et al., 2015), promotes conditioned place preference (Cole & Sumnall, 2003), and releases dopamine (Kehr et al., 2011) in the brain similar to, although not as robustly and strongly, as typical drugs of abuse such as methamphetamine. The risk of abuse of a substance with central-nervous system action is very generally associated with its dopamine stimulating properties.

There remains a need for further characterization of the enantiomers of MDMA and methods of optimizing effects of MDMA.

### SUMMARY OF THE INVENTION

The present invention provides for a composition including R-MDMA, S-MDMA, or specific (not 1:1 as in racemic MDMA) combinations of these two enantiomers of racemic MDMA as well as R-MDA, S-MDA, and a combination that is not 1:1 of R-MDA and S-MDA. These compositions can be beneficial in providing specific desired effects in an individual.

The present invention provides for a method of treating an individual, especially in substance-assisted psychotherapy by administering a composition of R-MDMA, S-MDMA, a combination that is not 1:1 of R-MDMA and S-MDMA, R-MDA, S-MDA, or a combination that is not 1:1 of R-MDA and S-MDA to the individual.

The present invention provides for a method of personalized medicine, by evaluating an individual who is in need of MDMA treatment and determining if there are characteristics of the individual present that would not be suitable for MDMA treatment, and administering a composition of R-MDMA, S-MDMA, a combination that is not 1:1 of R-MDMA and S-MDMA, R-MDA, S-MDA, or a combination that is not 1:1 of R-MDA and S-MDA to the individual.

The present invention provides for a method of reducing abuse of MDMA by an individual, by administering R-MDMA to the individual and thereby reducing abuse.

### DETAILED DESCRIPTION OF THE INVENTION

The present invention provides for methods of enhancing (qualitatively positively improving) the acute subjective (emotional, therapeutic) action of MDMA in the treatment for medical (mainly psychiatric) conditions while reducing adverse effects, i.e., optimizing the effects of MDMA. Thus, the benefit-harm profile of MDMA is improved to have a form of MDMA more suitable to treat individuals.

The benefit-harm profile of MDMA is improved by either increasing positive acute effects or reducing negative acute or sub-acute effects of MDMA by means of administering an enantiomer of MDMA (R-MDMA or S-MDMA) or a unique combination of these two enantiomers which is different from the 1:1 combination present in racemic MDMA. This allows modification of the MDMA effects to enhance its therapeutic benefits or/and to reduce its adverse effects. MDMA can be contraindicated in some subjects (for example due to cardiovascular side effects) and substance characteristics of R-or S-MDMA and slightly different from those of MDMA can be needed in some patients.

R-MDMA and S-MDMA can unexpectedly be more potent or have reduced toxicity than the racemic mixture of MDMA, and therefore it can be advantageous to administer an enantiomer as opposed to the racemic mixture.

Additionally, MDMA is metabolized in part to 3,4-methylenedioxyamphetamine (MDA) which is psychoactive. When using only R-MDMA or S-MDMA instead of racemic MDMA, only R-MDA and S-MDA is formed which can similarly result in advantages as opposed to the use of racemic MDMA and formation of racemic MDA as active metabolite.

Finally, the present invention can use the administration of the MDMA metabolites R-MDA or S-MDA or any non-1:1 combination thereof instead of using R-MDMA or S-MDMA to produce the desired effects in any of the methods described herein.

While R-MDMA can have a beneficial profile over MDMA in any subject, the benefits can even be enhanced in specific patients at a higher risk of adverse responses to racemic MDMA. In particular, R-MDMA has a pharmacological profile resulting in less stimulant-and more psychedelic-type effects compared with MDMA. R-MDMA can be a substitute for MDMA with reduced abuse liability, reduced cardiovascular adverse effects, and reduced risk of negative mood effects after its use. Similarly, S-MDMA can be advantageous in some patients based on its unique effects profile.

As further described below, R-MDMA can be used to provide more psychedelic-like effects compared with an equivalently psychoactive dose of racemic MDMA, produce significantly lower ratings of acute subjective “stimulation”, “high”, and “liking” compared with an equivalently psychoactive dose of racemic MDMA or S-MDMA, produce significantly lower acute autonomic stimulation (blood pressure, heart rate, rate-pressure product, and body temperature) than racemic MDMA or S-MDMA, produce significantly lower acute and sub-acute adverse effects than racemic MDMA or S-MDMA, and produce no or less lowered mood in the days 1-3 after its use compared with MDMA or S-MDMA. S-MDMA can be used to produce greater stimulation of oxytocin release compared with R-MDMA and similar to greater stimulation than MDMA.

The specific advantages are described herein an also directly investigated in human subjects in an ongoing clinical study described in EXAMPLE 1.

In general, R-MDMA can be administered in a dose of 20-400 mg, S-MDMA can be administered in a dose of 20-100 mg, R-MDA can be administered in a dose of 20-400 mg, and S-MDA can be administered in a dose of 20-100 mg.

The present invention provides for a method of treating an individual such as by inducing and enhancing positive acute and long-term therapeutic effects of MDMA in an individual, by administering R-MDMA, S-MDMA, or a unique combination thereof (not 1:1) to the individual, and treating the individual by enhancing a positive response compared with classic racemic MDMA.

The overall goal of the present invention is to improve or maintain the positive acute and therapeutic subjective effect response (i.e., induce positive acute subjective drug effects) of MDMA while reducing adverse effects typically associated with MDMA.

The method can be used for any indication of MDMA medication use and typically applies to psychiatric disorders including (but not limited to) post-traumatic stress disorder, substance use disorder, autism spectrum disorder, anxiety disorder, eating disorder, but can also include depression, obsessive-compulsive disorder, personality disorder, addictions, or it can be used even in couples therapy.

MDMA is an amphetamine derivative which, unlike prototypical amphetamines, predominantly enhances serotonergic neurotransmission via release of 5-HT through the SERT and it less potently also releases dopamine and norepinephrine through the dopamine transporter (DAT) and norepinephrine transporter (NET), respectively (Hysek et al., 2014b; Verrico et al., 2007). Furthermore, MDMA is known to trigger oxytocin release which may contribute to its effects to increase trust, prosociality, and enhanced empathy (Dumont et al., 2009; Hysek et al., 2014a). MDMA is therefore referred to as an “entactogen” or “empathogen”.

MDMA has been widely used for decades, especially among young people. Its popularity is likely associated with its emotional effects including feelings of well-being, positive mood, enhanced feelings of affection and connectedness to other people, increased openness, loss of anxiety, and feeling at peace (Schmid et al., 2014; Wardle & de Wit, 2014). These acute subjective effects of MDMA may also be helpful to assist psychotherapy.

MDMA is a racemic substance containing equal amounts (1:1 mixture) of the enantiomers S(+)-and R(−)-MDMA. Preclinical research indicates that S-MDMA mainly releases dopamine (DA)(Acquas et al., 2007; Hiramatsu & Cho, 1990; Murnane et al., 2010; Verrico et al., 2007), norepinephrine (NE) (Steele et al., 1987; Verrico et al., 2007), serotonin (5-HT) (Verrico et al., 2007), and oxytocin (Forsling et al., 2002) while R-MDMA may act more directly on 5-HT2A receptors (Fantegrossi et al., 2005; Nash et al., 1994) and be more hallucinogen-like (Murnane et al., 2009) and release prolactin (PRL)(Murnane et al., 2010).

Consistently, animal studies indicate that S-MDMA is more stimulant-like compared with R-MDMA while R-MDMA may be more psychedelic-like (Murnane et al., 2009) and have fewer adverse effects while still producing MDMA-typical effects at higher doses (Fantegrossi et al., 2003; Pitts et al., 2018; Young & Glennon, 2008). This more psychedelic-like profile of R-MDMA is considered advantageous in the present invention as it is linked to greater positive subjective effects and less addictive and stimulant type effects (including autonomic stimulation) compared with MDMA or S-MDMA. The clinical study described in EXAMPLE 1 as part of the present invention is used to evaluate and substantiate this positive effect profile of R-MDMA compared with MDMA across different doses of R-MDMA and in humans for the first time. EXAMPLE 1 includes a comparison of acute responses to specific doses of R-MDMA, S-MDMA, MDMA, and placebo in a cross-over study in healthy subjects. Based on preclinical data and this ongoing study in humans, the enantiomers of MDMA are proposed to have unique effect characteristics allowing their use in the treatment of medical disorders similar to MDMA but with an improved safety profile and including effects characteristics that are more suitable for specific disorders compared with the use of the standard racemic MDMA.

Specifically, in humans, R-MDMA administered at 125 mg or 250 mg, and in particular at the higher dose of 250 mg, is expected to result in greater psychedelic-type effects (5D-ASC total OAV score) compared with S-MDMA (125 mg). In contrast, 125 mg of S-MDMA is postulated to induce greater subjective stimulation (VAS) than 125 mg of R-MDMA.

Hyperthermia is a potentially fatal adverse effect of MDMA (Liechti, 2014a) and only S-MDMA but not R-MDMA induced hyperthermia in animals (Curry et al., 2018; Fantegrossi et al., 2003; Frau et al., 2013). Accordingly, in humans in EXAMPLE 1, 125 mg of R-MDMA is expected to produce less autonomic stimulation (blood pressure, heart rate, rate-pressure product, and body temperature) than 125 mg of MDMA or 125 mg of S-MDMA. Thus, the adverse cardiovascular effect profile and risk of hyperthermia associated with MDMA is expected to be improved by using R-MDMA in EXAMPLE 1.

In humans, in EXAMPLE 1, 125 mg of R-MDMA is expected to produce less adverse effects (LC total score) than 125 mg of MDMA or 125 mg of S-MDMA.

S-MDMA is more effective in depleting serotonin (Schmidt et al., 1987) and is more neurotoxic than R-MDMA when administered repeatedly at high doses (Frau et al., 2013). Accordingly, in humans, in EXAMPLE 1, S-MDMA is expected to result in lower mood day 1-3 after its administration compared with R-MDMA (BDI, SCL-90R, LC, AMRS in EXAMPLE 1) and similar to MDMA. Thus, the invention includes R-MDMA as a form of MDMA resulting in less negative subacute after effects also described as mid-week depression or blues (Liechti et al., 2001; Verheyden et al., 2002).

In humans, in EXAMPLE 1, S-MDMA (125 mg) is expected to produce greater stimulation of oxytocin release compared with R-MDMA (125 mg). This profile can be beneficial in certain disorders linked to lower oxytocin such as but not limited to autism spectrum disorder or hypopituitary malfunction/insufficiency and others.

One way of reducing the addictive property of a substance of abuse is by reducing its effects on the dopamine system and relatively enhancing serotonergic action. This approach is used by replacing MDMA with R-MDMA which is less dopaminergic than MDMA and stimulates the serotonin system to a greater extent. Racemic MDMA and both enantiomers have moderate reinforcing properties and S-MDMA is more potent than R-MDMA to maintain self-administration as tested in rhesus monkeys (Fantegrossi et al., 2002). S-MDMA but not R-MDMA reinstated amphetamine responding indicative of greater abuse-liability (McClung et al., 2010; Pitts et al., 2018). Accordingly, in humans in EXAMPLE 1, 125 mg of S-MDMA is expected to induce greater abuse-related subjective effects (VAS liking) than 125 mg of R-MDMA and similar to racemic MDMA.

The potentially therapeutic effect can also be distinct for each enantiomer (Pitts et al., 2018). For example, R-MDMA but not S-MDMA reduced conditioned fear when retention of extinction was assessed in a model of PTSD while S-MDMA reduced conditioned fear during extinction training but not thereafter (Curry et al., 2018). R-MDMA can therefore be particularly suitable for treatment of PTSD and maintaining therapeutic effects compared with S-MDMA.

R-MDMA also produced greater peak effects than S-MDMA on prosocial behaviors in monkeys (Pitts et al., 2017) and mice (Curry et al., 2018; Pitts et al., 2018). R-MDMA can therefore be particularly useful when aiming for maximal prosocial effects in humans and this difference is further evaluated also in the human study in EXAMPLE 1.

The metabolism (Meyer & Maurer, 2009) and pharmacokinetic profiles of R-and S-MDMA differ (Fantegrossi et al., 2009) in vitro and in animals, respectively. For example, in mice, the onset of interoceptive effects of S-MDMA and racemic MDMA were faster compared with R-MDMA and the duration of discriminative stimulus effects was shortest for R-MDMA (Fantegrossi et al., 2009). There was also enantiomeric interconversion in mice in that 3.5 and 9.9% of one enantiomer was converted to the other after administration of R-and S-MDMA, respectively (Fantegrossi et al., 2009). It is tested in EXAMPLE whether this is also the case in humans. Human studies have assessed the pharmacokinetics of R-and S-MDMA after administration of racemic MDMA but not after administration of the enantiomers (Steuer et al., 2016). After administration of racemic MDMA, plasma levels of the active metabolite S-MDA were higher and peaked earlier compared to R-MDA (Steuer et al., 2016). The concentration-time profiles of the parent R-and S-MDMA were relatively similar although there were small differences including slight changes in R/S ratios over time and after inhibition of CYP2D6 (Steuer et al., 2016). Differences in the pharmacokinetics of S- and R-MDMA are expected including differential effects of inhibition when MDMA is administered as racemate (Steuer et al., 2016). In humans, in EXAMPLE 1, there are likely distinct concentration-time profiles for S- and R-MDMA and S- and R-MDA which can also be beneficial in certain individuals.

Acute effects of S- and R-MDMA have never been validly examined in a human study. Therefore, the present study in EXAMPLE 1 as part of the present invention compares acute responses to R-MDMA, S-MDMA, ±MDMA, and placebo in a cross-over study in healthy subjects.

When designing a direct comparison of a racemic drug with its enantiomer as done in the present invention the dosing of each substance is key. Commonly, only one enantiomer of a racemic drug is active and it would therefore be dosed at half the dose in mg of the racemate. This is different for MDMA. In the case of MDMA, there is substantial evidence from animal studies and even human reports that both enantiomers are active and produce differential effects and are even reportedly needed to synergistically produce the full MDMA experience (Fantegrossi et al., 2003; Fantegrossi et al., 2002; Young & Glennon, 2008). Additionally, the effects and potency of racemic MDMA may likely be more distinct from what would be expected of the actions of its optical isomers (Young & Glennon, 2008).

Animal studies generally indicate that the two enantiomers act synergistically to produce the subjective effects of MDMA and that S-MDMA is mainly responsible for psychostimulation while R-MDMA can have fewer adverse effects and have greater prosocial effects (Fantegrossi et al., 2003; Pitts et al., 2018). R-MDMA is less potent than S-MDMA to produce locomotor stimulant and other behavioral effects in animals (Fantegrossi et al., 2003; Fantegrossi et al., 2005; Paulus & Geyer, 1992; Young & Glennon, 2008). S-MDMA is described to be mostly equipotent to racemic MDMA with regards to locomotor stimulant effects in rodents while R-MDMA is clearly less potent than racemic MDMA (Fantegrossi et al., 2003; Fantegrossi et al., 2005; Paulus & Geyer, 1992; Young & Glennon, 2008). Animal studies show mostly similar dose-response relationships for S-MDMA and racemic MDMA (Young & Glennon, 2008) indicating that the MDMA response is only partly due to S-MDMA and there are studies showing that the locomotor response produced by racemic MDMA is larger than would be predicted by simply adding the effects of the individual isomers (Fantegrossi et al., 2003). Lethal doses were also similar for S-MDMA and R-/S-MDMA in mice (Fantegrossi et al., 2003). Racemic MDMA is typically administered at a single dose of 80-125 mg (Liechti & Holze, 2021; Mithoefer et al., 2018; Vizeli & Liechti, 2017). Several studies in patients also used an additional dose of 40-62.5 mg MDMA 2 hours after the first dose resulting in a total dose of 120-187.5 mg (Mitchell et al., 2021; Oehen et al., 2013). 125 mg of racemic MDMA was selected as a representative and safe dose similar as in past studies (Vizeli & Liechti, 2017). Based on the animal data it is expected that S-MDMA and racemic MDMA are overall equipotent in inducing stimulant-type and adverse effects in humans. It is expected that a dose of S-MDMA of 125 mg is comparable to 125 mg of racemic MDMA with regards to subjective and autonomic stimulant-type effects but may not produce the full spectrum of empathogenic and prosocial effects of racemic MDMA. Theoretically, a dose of 62.5 mg S-MDMA would be equivalent to 125 mg of MDMA if only S-MDMA were active. However, because R-MDMA is also active and based on the synergy of the two enantiomers documented in animal studies (Fantegrossi et al., 2003) a higher dose of S-MDMA will very likely be needed to produce a full and characteristic response. Additionally, it is hypothesized that a dose of 125 mg of R-MDMA produces markedly fewer stimulant-type subjective effects compared with S-MDMA or racemic MDMA and a dose of 250 mg of R-MDMA produces greater empathogenic effects than 125 mg S-MDMA and similar to 125 mg of racemic MDMA. Because S-MDMA is mainly acting on the monoaminergic system and producing mainly the stimulant-type and adverse effects of MDMA (Pitts et al., 2018) it will not be dosed higher than 125 mg consistent also with its similar potency to MDMA in animal studies (Young & Glennon, 2008). However, R-MDMA will be dosed in an additional higher dose of 250 mg based on its reported lower potency (Young & Glennon, 2008) and reduced adverse effects profile and higher safety (Curry et al., 2018; Pitts et al., 2018) and to be able to assess its effects characteristics more fully.

A problem relating to using MDMA in the treatment of medical conditions is that MDMA has some abuse liability due to its amphetamine structure and pharmacology. Namely, MDMA releases dopamine (Kehr et al., 2011), which is associated with dependence. MDMA also releases serotonin (Kehr et al., 2011), which counteracts dependence (Suyama et al., 2016). Due to its combined dopaminergic and serotonergic properties, MDMA is considered a moderate reinforcer compared to methylphenidate, cocaine or nicotine, which are strong reinforcers (Liechti, 2014b). Nevertheless, abuse of MDMA can be a medical concern. R-MDMA is expected to be less addictive based on in vitro and animal data, but human data has been lacking and is generated herein.

A measure of abuse liability that can easily be measured is subjective drug liking (Jasinski, 2000; Jasinski & Krishnan, 2009a; Jasinski & Krishnan, 2009b). Subjective effects of drug high and drug liking are thought to be associated with abuse liability. In particular, higher drug-liking scores are predictors of greater abuse liability. #3875]. Thus, drug high and drug liking are expected to be lower after R-MDMA compared with S-MDMA as being evaluated in EXAMPLE 1.

MDMA and related substances increase blood pressure and, in some subjects, markedly (Hysek et al., 2011; Vizeli & Liechti, 2017). This can be a problem for subjects or patients with cardiovascular disease. MDMA-like substances with lower acute cardiovascular effects or an attenuated increase in blood pressure are warranted. R-MDMA is expected to exhibit an attenuated cardio-stimulant response based on its pharmacological profile. However, this has not yet been substantiated before the present invention.

Overall, characteristic of R-and S-MDMA versus MDMA are known based on in vitro studies and animal data. However, such data cannot substitute for human studies and the usefulness and applicability needs to be shown in humans as done within the present invention which includes the design and detailed plan of an experimental study experimentally testing and verifying and supporting the specific claims made for the use of R-and S-MDMA in humans.

The present invention provides for a method of personalized medicine, by evaluating an individual who is in need of MDMA treatment and determining if there are characteristics of the individual present that would not be suitable for MDMA treatment, and administering R-MDMA, S-MDMA, or a particular combination of the two to the individual. For example, if the individual has cardiac issues, it would be better to treat them with R-MDMA instead of MDMA. If the individual had experienced low mood after treatment with regular MDMA, treatment with R-MDMA would be advised. This approach provides maximum efficiency and minimizes toxicity to the individual.

The present invention provides for a method of reducing abuse of MDMA by an individual, by administering R-MDMA to the individual and thereby reducing abuse.

### Example 1 (Clinical Study Using R-MDMA and S-MDMA and Racemic MDMA in Healthy Subjects)

The overall goal of the present invention was to develop R-MDMA and S-MDMA as improved forms of MDMA to treat patients. A first step involves the first administration of R-MDMA and S-MDMA to healthy humans to study their acute effects and to compare these effects with those of racemic MDMA in healthy humans and at defined doses and demonstrating differences and potential advantages of R-MDMA and S-MDMA versus racemic MDMA. This is the first time the substances are administered in humans and the specific characteristics claimed in the present invention are verified and documented to be present in the species to be treated.

### Study Methods:

Study design: The present study compares the acute subjective, physiological and endocrine effects of S-MDMA (125 mg), R-MDMA (125 and 250 mg), MDMA (125 mg) using a placebo-controlled double-blind 5-period cross-over design in 24 healthy subjects. Conditions are 1.) R-MDMA 125 mg, 2). R-MDMA 250 mg, 3) S-MDMA 125 mg, 4) MDMA 125 mg, 5) placebo. Treatment order is counterbalanced. Washout periods between substance administrations are at least 10 days. Study days include a 10-hour study session with a subsequent single measurement (short visit) the next morning (24 hours after drug administration).

### Study Objectives:

Primary study hypothesis: R-MDMA at 125 mg or 250 mg, and in particular at the higher dose of 250 mg, is expected to result in greater psychedelic-type effects (5D-ASC total OAV score) compared with S-MDMA (125 mg). In contrast, 125 mg of S-MDMA induces greater subjective stimulation (VAS) than 125 mg of R-MDMA.

### Secondary Study Hypotheses

125 mg of S-MDMA produces greater autonomic stimulation (blood pressure, heart rate, rate-pressure product, and body temperature) than 125 mg of R-MDMA. 125 mg of S-MDMA produces more adverse effects (LC total score) than 125 mg of R-MDMA and similar to racemic MDMA. S-MDMA (125 mg) is expected to result in greater transient serotonin depletion and related untoward after effects. Specifically, S-MDMA results in lower mood day 1-3 after its administration compared with R-MDMA (BDI, SCL-90R, LC, AMRS) and similar to MDMA. S-MDMA (125 mg) is expected to produce greater stimulation of oxytocin release compared with R-MDMA (125 mg). There can be distinct concentration-time profiles for S- and R-MDMA and S- and R-MDA (descriptive endpoint). 125 mg of S-MDMA induces greater abuse-related subjective effects (VAS liking) than 125 mg of R-MDMA and similar to racemic MDMA.

Primary study endpoints: Subjective effects profile (5D-ASC, VAS stimulation).

Secondary study endpoints: Autonomic effects (blood pressure, heart rate, body temperature); Adverse effects (LC); Mood on day 1-3 after administration (BDI, SCL-90R, LC) and on day 3 (AMRS); Endocrine effects (cortisol, prolactin, oxytocin, vasopressin); Plasma concentrations of S-/R-MDMA and S-/R-MDA; Additional subjective effects (VAS, AMRS, SCQ).

Inclusion criteria: Age between 25 and 65 years. Understanding of the German language. Understanding the procedures and the risks that are associated with the study. Participants must be willing to adhere to the protocol and sign the consent form. Participants must be willing to refrain from taking illicit psychoactive substances during the study. Participants must be willing to drink only alcohol-free liquids and no coffee, black or green tea, or energy drink after midnight of the evening before the study session, as well as during the study day. Participants must be willing not to drive a traffic vehicle or to operate machines within 48 hours after substance administration. Willing to use double-barrier birth control throughout study participation. Body mass index between 18-29 kg/m².

Exclusion criteria: Chronic or acute medical condition. Current or previous major psychiatric disorder. Psychotic disorder in first-degree relatives, not including psychotic disorders secondary to an apparent medical reason, e.g., brain injury, dementia, or lesions of the brain. Hypertension (SBP>140/90 mmHg) or hypotension (SBP<85 mmHg). Illicit substance use (not including cannabis) more than 20 times or any time within the previous month. Pregnant or nursing women. Participation in another clinical trial (currently or within the last 30 days). Use of medications that may interfere with the effects of the study medications (any psychiatric medications). Tobacco smoking (>10 cigarettes/day). Consumption of alcoholic drinks (>15 drinks/week).

Participants: The target study sample size is 24 healthy subjects (12 males and 12 females). Drop-outs during the study are replaced to reach a final study sample of at least 20 subjects. Based on previous similar studies, about 36 potential participants are expected to be screened to include 24 subjects and about 4 drop-outs are expected during the study. Study duration is extended in case of increased drop outs or insufficient recruitment. Subjects are recruited via advertisement displayed on the website and bulletin boards of the University of Basel and the University Hospital of Basel. In case of insufficient recruitment, the study duration is extended.

Screening procedure: Physical health: Subjects are examined by a study physician. Basic health is ensured by general medical examination including medical history, physical examination, determination of body weight and blood chemistry and hematology analysis. Mental health: Subjects are screened using a semi-structured clinical interview for DSM-IV to exclude those with a personal axis I major psychiatric disorder (acute or past) or a history of drug dependence. Axis I major psychiatric disorders also include addiction disorders. In case of a psychiatric finding during the screening procedure, the study personnel provides the subject with information on where to find professional help and offer an appointment if necessary. History of drug use and drug screens: Occasional recreational drug use in the past is not an exclusion criterion if no adverse reactions occurred and if use was moderate and controlled. Subjects are excluded if they have had more than 20 past experiences in their life. Subjects are asked to abstain from any illicit drug use during the study and drug screens are performed during screening and randomly prior to sessions. Positive screens for stimulants, opioids or tranquilizers at the screening visit and/or at the study days result in study exclusion. Positive screens for Tetrahydrocannabinol (THC, cannabis) are recorded but do not result in study exclusion since THC consumption can be detected in urine for up to several weeks and THC consumption prior to a study day is unlikely to affect the outcome. Based on previous studies conducted in this study group, few if any positive screens are expected. Subjects are asked to abstain from excessive alcohol consumption between test sessions (less than 15 standard drinks/week) and to limit their use to one standard drink on the day prior to test sessions. Screening laboratory tests: A routine laboratory blood test is performed at the screening examination including creatinine, ALAT, hemoglobin, hematocrit, white blood cell count, red blood cell count, and platelet cell count. A urine drug screen and a pregnancy test in women will be performed.

Study procedures: The study takes place at the University Hospital Basel. Each of the test sessions last from 8:00 until 18:00 with a short visit the next morning (24 hours after application). An indwelling intravenous catheter is inserted into a subcutaneous vein of the forearm and baseline psychometric measures are obtained. Administration of the substance is at 9:00. Outcome measures (see below) are repeatedly assessed during the study session. Subjects are under continuous medical supervision until any alterations of consciousness have completely subsided and they leave the research facility.

Psychometric assessments: Visual Analog Scale (VAS): VASs is repeatedly used to assess subjective alterations in consciousness over time. Single scales are presented as 100 mm horizontal lines marked with “not at all” on the left and “extremely” on the right. The following VAS items are used: “any drug effect”, “good drug effect”, “bad drug effect”, “stimulated”, “liking”, “happy”, “content”, “talkative”, “open”, “trust”, “feeling close to others”, “anxiety”, “alteration of vision”, “alterations of hearing”, “sounds seem to influence what I see”, “alteration of sense of time”, “the boundaries between myself and my surroundings seem to blur”, “want to be with other people”, and “want to be alone”. Scales are administered before and repeatedly after substance administration and take approximately 2 minutes to fill out. The maximal ratings (Eₘₐₓ, 0-100) and areas under the effect-time curve (AUEC) are defined for each VAS and Eₘₐₓ and AUEC values compared between treatments using analysis of variance. 5-Dimensions of Altered States of Consciousness (5D-ASC): The 5 Dimensions of Altered States of Consciousness (5D-ASC) Scale is a questionnaire containing visual analog scales for 94 items (Studerus et al., 2010). The instrument contains five scales assessing mood, anxiety, derealization, depersonalization, changes in perception, auditory alterations, and reduced vigilance. The scale is well-validated (Studerus et al., 2010) and used internationally to evaluate effects of many other psychoactive substances. The overall psychedelic experience is reflected by the total 3D-ASC score. Each item of the scale is scored on a 0 -100 mm VAS. The attribution of the individual items to the subscales of the 5D-ASC is analyzed according to (Studerus et al., 2010). The 5D-ASC scale is administered once at the end of the session and subjects are instructed to retrospectively rate peak alterations that have been experienced during the study session. Adjective Mood Rating Scale (AMRS): The Adjective Mood Rating Scale (AMRS or EWL60S) is a 60-item Likert scale that allows repeated assessment of mood in 6 dimensions: Activation, inactivation, well-being, anxiety/depressed mood, extra-and introversion, and emotional excitability. The German EWL60S version is used. The AMRS consists of subscales measuring “activation”, “positive mood”, “extraversion”, “introversion”, “inactivation”, and “emotional excitability. The scale has previously been used to evaluate effects of many other psychoactive substances. Eₘₐₓ and AUEC scores will be defined for each subscale and compared between treatments using analysis of variance. The AMRS is administered repeatedly throughout the study session and once 3 days after each session. States of Consciousness Questionnaire (SCQ): In the SCQ, 100-items are rated on a six-point scale. Forty-three items embedded into this questionnaire comprise the Mystical Experience Questionnaire (MEQ) (MacLean et al., 2011) which is sensitive to the effects of psychedelic substances such as LSD. The 43 items provide scale scores for each of seven domains of mystical experiences: Internal unity (pure awareness, a merging with ultimate reality), external unity (unity of all things, all things are alive, all is one), sense of sacredness (reverence, sacred), noetic quality (encounter with ultimate reality, more real than everyday reality), transcendence of time and space, deeply felt positive mood (joy, peace, love), paradoxicality/ineffability (claim of difficulty in describing the experience in words). In this study, the four scale scores derived from the newly validated and revised 30-item MEQ are also used: Mystical, positive mood, transcendence of time and space, and ineffability (Barrett et al., 2015). The MEQ is an outcome measure for the mystical-type effects as this scale has become one of the standard measure in hallucinogen research. It complements the assessments with the 5D-ASC and is expected to be sensitive to the partial hallucinogenic effects of MDMA and to R-MDMA. Data on each domain scale is expressed as a percentage of the maximum possible score. The scale is administered once at the end of the session day. Spiritual Realm Questionnaire: The scale includes 11 main questions to be answered on a total of 65 sub-ordered visual rating scales. The scale is administered once at the end of the session. Beck Depression Inventory (BDI): The BDI consists of 21 questions developed to measure the severity of depression. The German BDI-II version is used as a self-assessment 3 days after substance administration and with reference to the last 3 days to assess any after effects on mood. Symptom-Check-List-90-R (SCL-90-R): The SCL-90R is a widely used psychological status symptom inventory. The German version is used 3 days after substance administration and with reference to the past 3 days to measure after effects on mood. Outcome measures are the global severity index the positive symptom distress index and the positive symptom total.

Autonomic measures: Blood pressure, heart rate, and body temperature are recorded at baseline and repeatedly throughout the session. Blood pressure (systolic and diastolic) and heart rate are measured with an automatic oscillometric device. Body temperature is measured with an ear thermometer. Eₘₐₓ is determined for each of the measures and for each study session. These Eₘₐₓ values are then compared between drug conditions using analysis of variance.

Adverse effects (list of complaints): The list of complaints (LC) consists of 66 items offering a global score measuring physical and general discomfort (Zerssen, 1976). The LC is administered before (baseline), at the end of the session to cover effects from 0-9 hours and 24 hours after substance administration to cover after effects from 9-24 hours. To assess adverse effects after 24 hours, subjects fill out an additional LC 3 days after the test session, covering adverse effects from 24-72 hours. Subjects are additionally be asked to report any adverse events during the sessions or/and between study sessions as assessed at the beginning of the next session and at the EOS visit. Global LC scores are summed up and compared between treatments using analysis of variance.

Drug plasma concentrations: To investigate individual substance exposure of participants, plasma levels of MDMA and MDA are measured repeatedly. The pharmacokinetics of racemic MDMA are well-known but it has not yet been tested how R-and S-MDMA and R-MDA and S-MDA plasma concentration-time curves are in the absence of the other enantiomer. Findings are described descriptively including maximal concentration (Cₘₐₓ), time to Cₘₐₓ, area under the concentration-time curve up to 24 hours (AUC₂₄) and elimination half-life values. MDMA and MDA plasma concentrations are determined in the Laboratory of the Division of Clinical Pharmacology and Toxicology at the Department of Biomedicine using a fully validated LC-MS/MS analytics method. Plasma concentration data are analyzed using non-compartmental methods.

Endocrine effects: Cortisol and prolactin: Cortisol and prolactin are determined as biological markers of serotonergic activity (Seifritz et al., 1996). Plasma concentrations are determined by routine immune assays. Oxytocin and vasopressin: MDMA increase circulating oxytocin (Hysek et al., 2014a) and vasopressin or its precursor copeptin (Simmler et al., 2011). These neurohormones are thought to mediate neurocognitive and adverse effects of MDMA and the role of R-versus S-MDMA remains to be explored. Oxytocin and vasopressin plasma levels are measured before and after substance administration. Concentrations are determined using immunoassays.

Drug products and doses: Analytically pure R-MDMA, S-MDMA and racemic MDMA are customer-synthesized in Switzerland. Capsules containing active substance (25 mg per capsule) plus matching placebo capsules (containing mannitol) are prepared by a Swissmedic-approved GMP facility in Switzerland. The products are produced according to GMP and tested for identity and content uniformity. Randomization, packaging, labelling, and quality control (QC) including stability tests for final products are performed. Randomization and blinding: Subjects and study personnel involved in supervising the session are blinded to treatment order. Order is balanced. The GMP facility performs the randomization.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intramuscular, and intranasal administration. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the individual in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Acquas E, Pisanu A, Spiga S, Plumitallo A, Zernig G, & Di Chiara G (2007). Differential effects of intravenous R, S-(+/−)-3,4-methylenedioxymethamphetamine (MDMA, Ecstasy) and its S(+)-and R(−)-enantiomers on dopamine transmission and extracellular signal regulated kinase phosphorylation (pERK) in the rat nucleus accumbens shell and core. J Neurochem 102:121-132.
2. Barrett F S, Johnson M W, & Griffiths R R (2015). Validation of the revised Mystical Experience Questionnaire in experimental sessions with psilocybin. J Psychopharmacol 29:1182-1190.
3. Cole J C, & Sumnall H R (2003). The pre-clinical behavioural pharmacology of 3,4-methylenedioxymethamphetamine (MDMA). Neurosci Biobehav Rev 27:199-217.
4. Creehan K M, Vandewater S A, & Taffe M A (2015). Intravenous self-administration of mephedrone, methylone and MDMA in female rats. Neuropharmacology 92C: 90-97.
5. Curry D W, Young M B, Tran A N, Daoud G E, & Howell L L (2018). Separating the agony from ecstasy: R(−)-3,4-methylenedioxymethamphetamine has prosocial and therapeutic-like effects without signs of neurotoxicity in mice. Neuropharmacology 128:196-206.
6. Danforth A L, Grob C S, Struble C, Feduccia A A, Walker N, Jerome L, Yazar-Klosinski B, & Emerson A (2018). Reduction in social anxiety after MDMA-assisted psychotherapy with autistic adults: a randomized, double-blind, placebo-controlled pilot study. Psychopharmacology (Berl) 235:3137-3148.
7. Danforth A L, Struble C M, Yazar-Klosinski B, & Grob C S (2016). MDMA-assisted therapy: A new treatment model for social anxiety in autistic adults. Prog Neuropsychopharmacol Biol Psychiatry 64:237-249.
8. Dolder P C, Muller F, Schmid Y, Borgwardt S J, & Liechti M E (2018). Direct comparison of the acute subjective, emotional, autonomic, and endocrine effects of MDMA, methylphenidate, and modafinil in healthy subjects. Psychopharmacology (Berl) 235:467-479.
9. Dumont G J, Sweep F C, van der Steen R, Hermsen R, Donders A R, Touw D J, van Gerven J M, Buitelaar J K, & Verkes R J (2009). Increased oxytocin concentrations and prosocial feelings in humans after ecstasy (3,4-methylenedioxymethamphetamine) administration. Soc Neurosci 4:359-366.
10. Fantegrossi W E, Godlewski T, Karabenick R L, Stephens J M, Ullrich T, Rice K C, & Woods J H (2003). Pharmacological characterization of the effects of 3,4-methylenedioxymethamphetamine (“ecstasy”) and its enantiomers on lethality, core temperature, and locomotor activity in singly housed and crowded mice. Psychopharmacology (Berl) 166:202-211.
11. Fantegrossi W E, Kiessel C L, De la Garza R, 2nd, & Woods J H (2005). Serotonin synthesis inhibition reveals distinct mechanisms of action for MDMA and its enantiomers in the mouse. Psychopharmacology (Berl) 181:529-536.
12. Fantegrossi W E, Murai N, Mathuna B O, Pizarro N, & de la Torre R (2009). Discriminative stimulus effects of 3,4-methylenedioxymethamphetamine and its enantiomers in mice: pharmacokinetic considerations. J Pharmacol Exp Ther 329: 1006-1015.
13. Fantegrossi W E, Ullrich T, Rice K C, Woods J H, & Winger G (2002). 3,4-Methylenedioxymethamphetamine (MDMA, “ecstasy”) and its stereoisomers as reinforcers in rhesus monkeys: serotonergic involvement. Psychopharmacology (Berl) 161:356-364.
14. Forsling M L, Fallon J K, Shah D, Tilbrook G S, Cowan D A, Kicman A T, & Hutt A J (2002). The effect of 3,4-methylenedioxymethamphetamine (MDMA, “ecstasy”) and its metabolites on neurohypophysial hormone release from the isolated rat hypothalamus. Br J Pharmacol 135:649-656.
15. Frau L, Simola N, Plumitallo A, & Morelli M (2013). Microglial and astroglial activation by 3,4-methylenedioxymethamphetamine (MDMA) in mice depends on S(+) enantiomer and is associated with an increase in body temperature and motility. J Neurochem 124:69-78.
16. Hiramatsu M, & Cho A K (1990). Enantiomeric differences in the effects of 3,4-methylenedioxymethamphetamine on extracellular monoamines and metabolites in the striatum of freely-moving rats: an in vivo microdialysis study. Neuropharmacology 29:269-275.
17. Holze F, Vizeli P, Muller F, Ley L, Duerig R, Varghese N, Eckert A, Borgwardt S, & Liechti ME (2020). Distinct acute effects of LSD, MDMA, and D-amphetamine in healthy subjects. Neuropsychopharmacology 45:462-471.
18. Hysek C M, Schmid Y, Simmler L D, Domes G, Heinrichs M, Eisenegger C, Preller K H, Quednow B B, & Liechti M E (2014a). MDMA enhances emotional empathy and prosocial behavior. Soc Cogn Affect Neurosci 9:1645-1652.
19. Hysek C M, Simmler L D, Ineichen M, Grouzmann E, Hoener M C, Brenneisen R, Huwyler J, & Liechti M E (2011). The norepinephrine transporter inhibitor reboxetine reduces stimulant effects of MDMA (“ecstasy”) in humans. Clin Pharmacol Ther 90:246-255.
20. Hysek C M, Simmler L D, Schillinger N, Meyer N, Schmid Y, Donzelli M, Grouzmann E, & Liechti M E (2014b). Pharmacokinetic and pharmacodynamic effects of methylphenidate and MDMA administered alone and in combination. Int J Neuropsychopharmacol 17:371-381.
21. Jasinski D R (2000). An evaluation of the abuse potential of modafinil using methylphenidate as a reference. J Psychopharmacol 14:53-60.
22. Jasinski D R, & Krishnan S (2009a). Abuse liability and safety of oral lisdexamfetamine dimesylate in individuals with a history of stimulant abuse. J Psychopharmacol 23: 419-427.
23. Jasinski D R, & Krishnan S (2009b). Human pharmacology of intravenous lisdexamfetamine dimesylate: abuse liability in adult stimulant abusers. J Psychopharmacol 23:410-418.
24. Kehr J, Ichinose F, Yoshitake S, Goiny M, Sievertsson T, Nyberg F, & Yoshitake T (2011). Mephedrone, compared to MDMA (ecstasy) and amphetamine, rapidly increases both dopamine and serotonin levels in nucleus accumbens of awake rats. Br J Pharmacol 164:1949-1958.
25. Liechti M E (2014a). Effects of MDMA on body temperature in humans. Temperature 1:179-187.
26. Liechti M E (2014b). Novel psychoactive substances (designer drugs): overview and pharmacology of modulators of monoamine signalling. Swiss Med Weekly 144: w 14043.
27. Liechti M E, Gamma A, & Vollenweider F X (2001). Gender differences in the subjective effects of MDMA. Psychopharmacology (Berl) 154:161-168.
28. Liechti M E, & Holze F (2021). Dosing psychedelics and MDMA. Curr Topics Behav Neurosci doi: 10.1007/7854_2021_270.
29. MacLean K A, Johnson M W, & Griffiths R R (2011). Mystical experiences occasioned by the hallucinogen psilocybin lead to increases in the personality domain of openness. J Psychopharmacol 25:1453-1461.
30. McClung J, Fantegrossi W, & Howell L L (2010). Reinstatement of extinguished amphetamine self-administration by 3,4-methylenedioxymethamphetamine (MDMA) and its enantiomers in rhesus monkeys. Psychopharmacology (Berl) 210: 75-83.
31. Meyer M R, & Maurer H H (2009). Enantioselectivity in the methylation of the catecholic phase I metabolites of methylenedioxy designer drugs and their capability to inhibit catechol-O-methyltransferase-catalyzed dopamine 3-methylation. Chem Res Toxicol 22:1205-1211.
32. Mitchell J M, Bogenschutz M, Lilienstein A, Harrison C, Kleiman S, Parker-Guilbert K, Otalora G M, Garas W, Paleos C, Gorman I, Nicholas C, Mithoefer M, Carlin S, Poulter B, Mithoefer A, Quevedo S, Wells G, Klaire S S, van der Kolk B, Tzarfaty K, Amiaz R, Worthy R, Shannon S, Woolley J D, Marta C, Gelfand Y, Hapke E, Amar S, Wallach Y, Brown R, Hamilton S, Wang J B, Coker A, Matthews R, de Boer A, Yazar-Klosinski B, Emerson A, & Doblin R (2021). MDMA-assisted therapy for severe PTSD: a randomized, double-blind, placebo-controlled phase 3 study. Nat Med 27:1025-1033.
33. Mithoefer M C, Feduccia A A, Jerome L, Mithoefer A, Wagner M, Walsh Z, Hamilton S, Yazar-Klosinski B, Emerson A, & Doblin R (2019). MDMA-assisted psychotherapy for treatment of PTSD: study design and rationale for phase 3 trials based on pooled analysis of six phase 2 randomized controlled trials. Psychopharmacology (Berl).
34. Mithoefer M C, Mithoefer A T, Feduccia A A, Jerome L, Wagner M, Wymer J, Holland J, Hamilton S, Yazar-Klosinski B, Emerson A, & Doblin R (2018). 3,4-methylenedioxymethamphetamine (MDMA)-assisted psychotherapy for post-traumatic stress disorder in military veterans, firefighters, and police officers: a randomised, double-blind, dose-response, phase 2 clinical trial. Lancet Psychiatry 5:486-497.
35. Mithoefer M C, Wagner M T, Mithoefer A T, Jerome I, & Doblin R (2010). The safety and efficacy of ±3,4-methylenedioxymethamphetamine-assisted psychotherapy in subjects with chronic, treatment-resistant posttraumatic stress disorder: the first randomized controlled pilot study. J Psychopharmacol 25:439-452.
36. Murnane K S, Fantegrossi W E, Godfrey J R, Banks M L, & Howell L L (2010). Endocrine and neurochemical effects of 3,4-methylenedioxymethamphetamine and its stereoisomers in rhesus monkeys. J Pharmacol Exp Ther 334:642-650.
37. Murnane K S, Murai N, Howell L L, & Fantegrossi W E (2009). Discriminative stimulus effects of psychostimulants and hallucinogens in S(+)-3,4-methylenedioxymethamphetamine (MDMA) and R(−)-MDMA trained mice. J Pharmacol Exp Ther 331:717-723.
38. Nash J F, Roth B L, Brodkin J D, Nichols D E, & Gudelsky G A (1994). Effect of the R(−) and S(+) isomers of MDA and MDMA on phosphatidyl inositol turnover in cultured cells expressing 5-HT2A or 5-HT2C receptors. Neurosci Lett 177:111-115.
39. Oehen P, Traber R, Widmer V, & Schnyder U (2013). A randomized, controlled pilot study of MDMA (±3,4-methylenedioxymethamphetamine)-assisted psychotherapy for treatment of resistant, chronic post-traumatic stress disorder (PTSD). J Psychopharmacol 27:40-52.
40. Paulus M P, & Geyer M A (1992). The effects of MDMA and other methylenedioxy-substituted phenylalkylamines on the structure of rat locomotor activity. Neuropsychopharmacology 7:15-31.
41. Pitts E G, Curry D W, Hampshire K N, Young M B, & Howell L L (2018). (+/−)-MDMA and its enantiomers: potential therapeutic advantages of R(−)-MDMA. Psychopharmacology (Berl) 235:377-392.
42. Pitts E G, Minerva A R, Chandler E B, Kohn J N, Logun M T, Sulima A, Rice K C, & Howell L L (2017). 3,4-Methylenedioxymethamphetamine increases affiliative behaviors in squirrel monkeys in a serotonin 2A receptor-dependent manner. Neuropsychopharmacology 42:1962-1971.
43. Schmid Y, Hysek C M, Simmler L D, Crockett M J, Quednow B B, & Liechti M E (2014). Differential effects of MDMA and methylphenidate on social cognition. J Psychopharmacol 28:847-856.
44. Schmidt C J, Levin J A, & Lovenberg W (1987). In vitro and in vivo neurochemical effects of methylenedioxymethamphetamine on striatal monoaminergic systems in the rat brain. Biochem Pharmacol 36:747-755.
45. Seifritz E, Baumann P, Muller M J, Annen O, Amey M, Hemmeter U, Hatzinger M, Chardon F, & Holsboer-Trachsler E (1996). Neuroendocrine effects of a 20-mg citalopram infusion in healthy males: a placebo-controlled evaluation of citalopram as 5-HT function probe. Neuropsychopharmacology 14:253-263.
46. Simmler L D, Hysek C M, & Liechti M E (2011). Sex differences in the effects of MDMA (ecstasy) on plasma copeptin in healthy subjects. J Clin Endocrinol Metab 96:2844-2850.
47. Steele T D, Nichols D E, & Yim G K (1987). Stereochemical effects of 3,4-methylenedioxymethamphetamine (MDMA) and related amphetamine derivatives on inhibition of uptake of [3H]monoamines into synaptosomes from different regions of rat brain. Biochem Pharmacol 36:2297-2303.
48. Steuer A E, Schmidhauser C, Tingelhoff E H, Schmid Y, Rickli A, Kraemer T, & Liechti M E (2016). Impact of cytochrome P450 2D6 function on the chiral blood plasma pharmacokinetics of 3,4-Methylenedioxymethamphetamine (MDMA) and its phase I and II metabolites in humans. PLOS One 11: e0150955.
49. Studerus E, Gamma A, & Vollenweider F X (2010). Psychometric evaluation of the altered states of consciousness rating scale (OAV). PLOS One 5: e12412.
50. Suyama J A, Sakloth F, Kolanos R, Glennon R A, Lazenka M F, Negus S S, & Banks M L (2016). Abuse-related neurochemical effects of para-substituted methcathinone analogs in rats: microdialysis studies of nucleus accumbens dopamine and serotonin. J Pharmacol Exp Ther 356:182-190.
51. Verheyden S L, Hadfield J, Calin T, & Curran H V (2002). Sub-acute effects of MDMA (+/−3,4-methylenedioxymethamphetamine, “ecstasy”) on mood: evidence of gender differences. Psychopharmacology (Berl) 161:23-31.
52. Verrico C D, Miller G M, & Madras B K (2007). MDMA (ecstasy) and human dopamine, norepinephrine, and serotonin transporters: implications for MDMA-induced neurotoxicity and treatment. Psychopharmacology 189:489-503.
53. Vizeli P, & Liechti M E (2017). Safety pharmacology of acute MDMA administration in healthy subjects. J Psychopharmacol 31:576-588.
54. Wardle M C, & de Wit H (2014). MDMA alters emotional processing and facilitates positive social interaction. Psychopharmacology (Berl) 231:4219-4229.
55. Young R, & Glennon R A (2008). MDMA (N-methyl- 3,4-methylenedioxyamphetamine) and its stereoisomers: Similarities and differences in behavioral effects in an automated activity apparatus in mice. Pharmacol Biochem Behav 88:318-331.
56. Zerssen D V (1976) Die Beschwerden-Liste. Münchener Informationssystem. Psychis: München.

What is claimed is:

1. A composition comprising an effective amount of a compound chosen from the group consisting of R-MDMA, S-MDMA, a combination that is not 1:1 of R-MDMA and S-MDMA, R-MDA, S-MDA, and a combination that is not 1:1 of R-MDA and S-MDA.

2. The composition of claim 1, wherein the compound is R-MDMA and induces more psychedelic-type effects in an individual compared with an equivalently psychoactive dose of racemic MDMA.

3. The composition of claim 1, wherein the compound is R-MDMA and produces lower abuse-related subjective effects including lower scale ratings of “stimulation”, “high”, and “liking” compared with an equivalently psychoactive dose of racemic MDMA.

4. The composition of claim 1, wherein the compound is R-MDMA and produces lower autonomic stimulation including lower blood pressure, heart rate, rate-pressure product, and body temperature compared with an equivalently psychoactive dose of racemic MDMA.

5. The composition of claim 1, wherein the compound is R-MDMA and produces lower acute and sub-acute adverse effects compared with an equivalently psychoactive dose of racemic MDMA.

6. The composition of claim 1, wherein the compound is R-MDMA and produces no or less lowered mood in the days 1-3 after its use compared with an equivalently psychoactive dose of racemic MDMA or S-MDMA.

7. The composition of claim 1, wherein the compound is S-MDMA and produces greater stimulation of oxytocin release compared with an equivalently psychoactive dose of racemic MDMA or R-MDMA.

8. A method of treating an individual, including the steps of:
administering a composition chosen from the group consisting of R-MDMA, S-MDMA, a combination that is not 1:1 of R-MDMA and S-MDMA, R-MDA, S-MDA, and a combination that is not 1:1 of R-MDA and S-MDA to the individual; and
treating the individual.

9. The method of claim 8, wherein said treating step is further defined as inducing positive acute effects in the individual.

10. The method of claim 8, further including the step of reducing adverse effects associated with MDMA.

11. The method of claim 8, wherein the individual has a psychiatric disorder chosen from the group consisting of post-traumatic stress disorder, substance use disorder, autism spectrum disorder, anxiety disorder, eating disorder, depression, obsessive-compulsive disorder, personality disorder, and addictions.

12. The method of claim 8, wherein the composition is administered for couples therapy.

13. The method of claim 8, wherein the composition is R-MDMA and is administered in a dose of 20-400 mg.

14. The method of claim 8, wherein the composition is S-MDMA and is administered in a dose of 20-100 mg.

15. The method of claim 8, wherein the composition is R-MDA and is administered in a dose of 20-400 mg.

16. The method of claim 8, wherein the composition is S-MDA and is administered in a dose of 20-100 mg.

17. A method of personalized medicine, including the steps of:
evaluating an individual who is in need of MDMA treatment and determining if there are characteristics of the individual present that would not be suitable for MDMA treatment; and
administering a composition chosen from the group consisting of R-MDMA, S-MDMA, a combination that is not 1:1 of R-MDMA and S-MDMA, R-MDA, S-MDA, and a combination that is not 1:1 of R-MDA and S-MDA to the individual.

18. The method of claim 17, wherein said evaluating step is further defined as determining if the individual has cardiac issues, and the composition is R-MDMA.

19. The method of claim 17, wherein said evaluating step is further defined as determining if the individual has previously experienced low mood after treatment with MDMA, and the composition is R-MDMA.

20. A method of reducing abuse of MDMA by an individual, including the steps of:
administering R-MDMA to the individual and thereby reducing abuse.
